(19) United States
(12) Patent Application Publication
(10) Pub. No.: US 2021/0323932 A1
(43) Pub. Date: Oct. 21, 2021

(54) COMPOSITIONS FOR INHIBITION OF HISTONE DEMETHYLASES AND/OR INDUCTION OF TUMOR SUPPRESSOR HEXIM1

(71) Applicant: CASE WESTERN RESERVE UNIVERSITY, Cleveland, OH (US)

(72) Inventors: Monica Montano, Shaker Heights, OH (US); Matthew C. Lawes, Shaker Heights, OH (US)

(21) Appl. No.: 17/234,343

(22) Filed: Apr. 19, 2021

Related U.S. Application Data
(60) Provisional application No. 63/011,502, filed on Apr. 17, 2020.

(51) Int. Cl. C07D 241/44; A61P 35/00

(52) U.S. Cl. CPC C07D 241/44; A61P 35/00

(57) ABSTRACT

Compounds for use in treating cancer include inhibitors of KDM5B and/or inducers of HEXIM1 and/or p21 are described herein.

### RELATED APPLICATION

This application claims priority from U.S. Provisional Application No. 63/011,502, filed Apr. 17, 2020, the subject matter of which is incorporated herein by reference in its entirety.

### GOVERNMENT FUNDING

This invention was made with government support under Grant No. CA195558 awarded by the National Institute of Health, and Grant No. IK2 BX002683, from the Department of Veteran Affairs. The United States government has certain rights to the invention.

### BACKGROUND

Hexamethylene bis-acetamide (HMBA) is a small molecule that has been investigated due to its potent anti-cancer and cell differentiation activities. Increased HEXIM1 expression caused differentiation and inhibited proliferation and metastasis of cancer cells. Historically HEXIM1 has been experimentally induced by the hybrid polar compound HMBA. However, HMBA failed in Phase II clinical trials because of a toxicity, thrombocytopenia, and its short half-life requiring infusion at a high dosage. HMBA induces terminal differentiation via upregulation of HMBA-inducible protein 1 (HEXIM1), albeit at mM levels. HEXIM1 also plays a central role in the anti-cancer activities of another category of therapeutics, BET inhibitors. Thus, HEXIM1 induction is an intriguing therapeutic approach to cancer treatment, but requires better chemical tools than HMBA. Pharmacological induction of HEXIM1 expression previously lacked any known target for direct interaction with HEXIM1 inducing compounds.

### SUMMARY

Embodiments described herein relate to compounds that inhibit KDM5B and/or induce expression of HEXIM1 and/or p21 and that can be administered to a subject at therapeutically effective amounts to treat cancer in the subject. The cancer can include, for example, prostate and breast cancer, such as triple negative breast cancer.

In some embodiments, the compound can have the following formula:

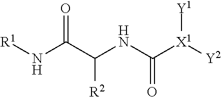

or a pharmaceutically acceptable salt, tautomer, or solvate thereof;

wherein X¹ is N(H) or C(H); and

R¹, R², Y¹, and Y² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, or a substituted or unsubstituted heterocyclyl, and wherein the compound inhibits KDM5B and/or induces expression of HEXIM1 and/or p21 in cancer cells of a(the) subject.

In some embodiments, R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is a halogen, alkyl, alkoxy, or hydroxy.

In other embodiments, R¹ is an alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, and wherein R⁷ is a halogen, alkyl, alkoxy, or hydroxy.

In some embodiments, R² is an alkyl, alkylene-aryl, aryl, alkylene-cycloalkyl, cycloalkyl, alkylene-heterocyclyl, or heterocyclyl, each of which is optionally substituted with one or more R⁸, and wherein R⁸ is a halogen, alkyl, alkoxy, or hydroxy.

In some embodiments, Y¹ and Y² are linked to form a cyclic or polycyclic ring, wherein the ring is an aryl, a heteroaryl, a cycloalkyl, or a heterocyclyl, each of which is optionally substituted with one or more R⁹, and wherein R⁹ is a halogen, alkyl, alkoxy, or hydroxy.

In other embodiments, Y¹ and Y² are linked to form a cycloalkane, cycloalkenyl, phenylene, biphenylene, pentalene, indene, naphthalene, auzlene, indacenepyrrole, imidazole, pyrazole, indazole, purine, pyridine, pyrimidine, pyridazine, quinolizine, isonquinoline, phthalazine, naphthyridine, quinoxaline, quinazoline, cinnoline, pteridine, indolizine, indole, carbazole, carboline, acridine, phenanthridine, phenazine, or phenanthroline, each of which is optionally substituted with one or more R¹⁰, and wherein each R¹⁰ is a hydrogen, halogen, alkyl, alkoxy, or hydroxy.

In some embodiments, the compound can have the following formula:

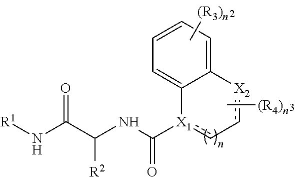

or a pharmaceutically acceptable salt, tautomer, or solvate thereof;

wherein X¹ and X² are each independently N(H) or C(H);

the dashed line is an optional bond;

R¹ and R² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof;

R³ and R⁴ are individually absent or are selected from a hydrogen, halogen, alkyl, alkoxy, or hydroxy;

n is 0 or 1;

n² is 0, 1, 2, or 3; and

n³ is 0, 1, 2, or 3.

In some embodiments, R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is a halogen, alkyl, alkoxy, or hydroxy.

In other embodiments, R¹ is a alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, and wherein R⁷ is a halogen, alkyl, alkoxy, or hydroxy.

In some embodiments, R² is an alkyl, alkylene-aryl, aryl, alkylene-cycloalkyl, cycloalkyl, alkylene-heterocyclyl, or heterocyclyl, each of which is optionally substituted with one or more R⁸, wherein R⁸ is a halogen, alkyl, alkoxy, or hydroxy.

In other embodiments, the compound is selected from:

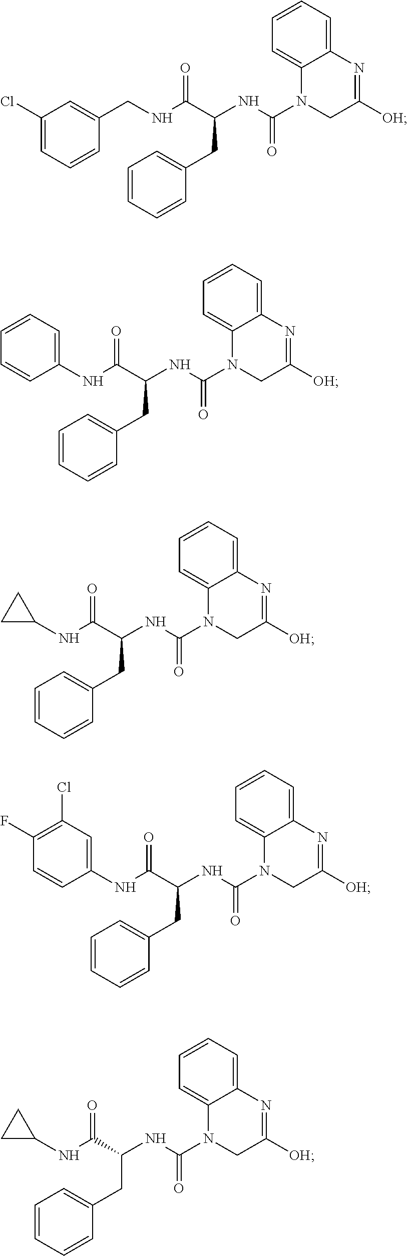 [SMILES: O=C(NCc1cccc(Cl)c1)[C@H](Cc1ccccc1)NC(=O)N1CC(O)=Nc2ccccc21 ; O=C(Nc1ccccc1)[C@H](Cc1ccccc1)NC(=O)N1CC(O)=Nc2ccccc21 ; O=C(NC1CC1)[C@H](Cc1ccccc1)NC(=O)N1CC(O)=Nc2ccccc21 ; O=C(Nc1ccc(F)c(Cl)c1)[C@H](Cc1ccccc1)NC(=O)N1CC(O)=Nc2ccccc21 ; O=C(NC1CC1)[C@@H](Cc1ccccc1)NC(=O)N1CC(O)=Nc2ccccc21]

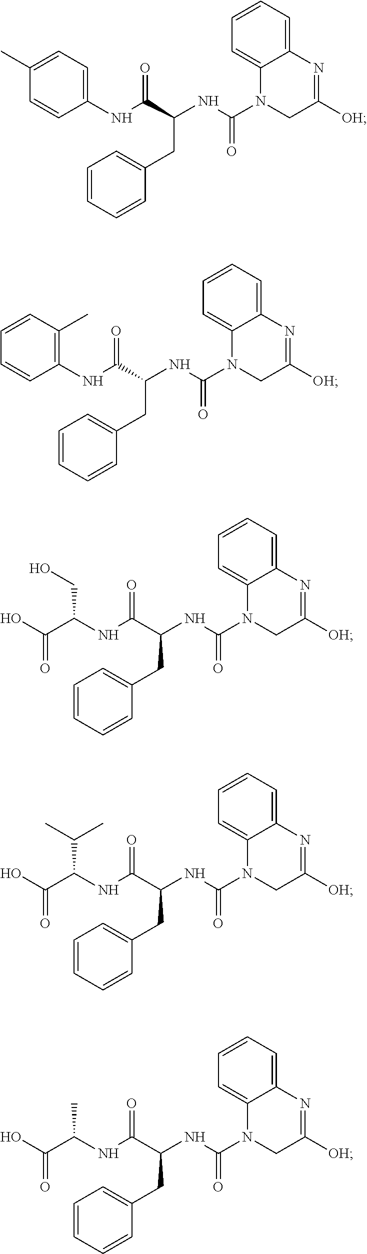 [SMILES: Cc1ccc(NC(=O)[C@H](Cc2ccccc2)NC(=O)N2CC(O)=Nc3ccccc32)cc1 ; Cc1ccccc1NC(=O)[C@@H](Cc1ccccc1)NC(=O)N1CC(O)=Nc2ccccc21 ; O=C(O)[C@H](CO)NC(=O)[C@H](Cc1ccccc1)NC(=O)N1CC(O)=Nc2ccccc21 ; CC(C)[C@H](NC(=O)[C@H](Cc1ccccc1)NC(=O)N1CC(O)=Nc2ccccc21)C(=O)O ; C[C@H](NC(=O)[C@H](Cc1ccccc1)NC(=O)N1CC(O)=Nc2ccccc21)C(=O)O]

or a pharmaceutically acceptable salt, tautomer, or solvate thereof.

In some embodiments, the compounds described herein can be administered to a subject with cancer at an amount effective to inhibit KDM5B, induce HEXIM1 expression, and/or induce p21 expression.

### BRIEF DESCRIPTION OF THE DRAWINGS

FIG. 1 illustrates a graph showing known competitive inhibitor KDOAM25 at its approximate IC₅₀ of 50 nM against 15, 65 or 165 μM αKG.

FIG. 2 illustrates a graph showing the induction of HEXIM1 protein in MDA-MB-231 Triple Negative Breast Cancer (TNBC) cells using compounds 5, 8, or 10 at 0.5 or 5 nM.

FIG. 3 illustrate a graph showing only a subset of the compounds are effective at also inducing p21 (the marker of quiescence/G₀), in MDA-MB-231 and other TNBC cell lines.

FIG. 4 illustrates a graph showing the overall impact of HEXIM1 induction combined with an effective induction of p21 can be seen in the results of a proliferation assay using TNBC MDA-MB-231 and all the members of the lead chemical series at 0.5 and 5 nM doses.

FIG. 5 illustrates a graph comparing some members of the lead compound series against much higher doses of KDOAM25.

FIG. 6 illustrates a graph showing the result using a ChIP assay on the −3179/−2595 region of the HEXIM1 promoter and shows that inhibitor OSX44 likewise causes a particularly strong accumulation of H3K4me3 on the promoter.

FIG. 7 is a graph illustrating prostate cancer cell line DU145 cells treated with 0.5 nM, 5 nM or 50 nM of either compound KDOAM25 (IC50˜42 nM) or compound 8.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C5 alkyls, C4 alkyls, C3 alkyls, C2 alkyls and C1 alkyl (i.e., methyl). A C₁-C₆ alkyl includes all moieties described above for C₁-C₅ alkyls but also includes C6 alkyls. A C₁-C₁₀ alkyl includes all moieties described above for C₁-C₅ alkyls and C1-C6 alkyls, but also includes C7, C8, C9 and C10 alkyls. Similarly, a C₁-C₁₂ alkyl includes all the foregoing moieties, but also includes C11 and C12 alkyls. Non-limiting examples of C1-C12 alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C2-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}-R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula -R_{b}-R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}-R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}-Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}-Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}-Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}-R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}-R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}-R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{B}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

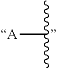

indicates that the chemical entity “A” is bonded to another chemical entity via the point of attachment bond. Furthermore, the specific point of attachment to the non-depicted chemical entity can be specified by inference. For example, the compound

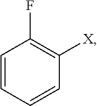

wherein X is

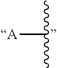

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In some embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

Embodiments described herein relate to compounds and methods of inhibiting KDM5B and/or inducing HEXIM1 and/or p21 expression in cancer cells and to their use in treating cancer in a subject in need thereof.

In some embodiments, the compound can have the following formula:

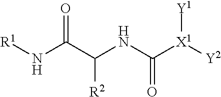

or a pharmaceutically acceptable salt, tautomer, or solvate thereof;

wherein X¹ is N(H) or C(H); and

R¹, R², Y¹, and Y² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, or a substituted or unsubstituted heterocyclyl.

In some embodiments, R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, wherein R⁷ is a halogen, alkyl, alkoxy, or hydroxy.

In some embodiments, R¹ is a alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, wherein R⁷ is a halogen, alkyl, alkoxy, or hydroxy.

In some embodiments, R² is an alkyl, alkylene-aryl, aryl, alkylene-cycloalkyl, cycloalkyl, alkylene-heterocyclyl, or heterocyclyl, each of which is optionally substituted with one or more R⁸, wherein R⁸ is halogen, alkyl, alkoxy, or hydroxy.

In some embodiments, Y¹ and Y² are linked to form a cyclic or polycyclic ring, wherein the ring is an aryl, a heteroaryl, a cycloalkyl, or a heterocyclyl, each of which is optionally substituted with one or more R⁹, wherein R⁹ is a halogen, alkyl, alkoxy, or hydroxy.

In other embodiments, Y¹ and Y² are linked to form a cycloalkane, cycloalkenyl, phenylene, biphenylene, pentalene, indene, naphthalene, auzlene, indacenepyrrole, imidazole, pyrazole, indazole, purine, pyridine, pyrimidine, pyridazine, quinolizine, isonquinoline, phthalazine, naphthyridine, quinoxaline, quinazoline, cinnoline, pteridine, indolizine, indole, carbazole, carboline, acridine, phenanthridine, phenazine, or phenanthroline, each of which is optionally substituted with one or more R¹⁰, and wherein each R¹⁰ is a hydrogen, halogen, alkyl, alkoxy, or hydroxy.

In other embodiments, the compound can have the following formula:

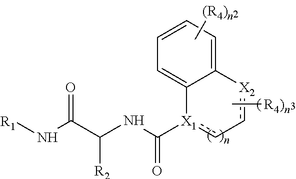

or a pharmaceutically acceptable salt, tautomer, or solvate thereof;

wherein X¹ and X² are each independently N or C(H);

the dashed line is an optional bond;

R¹ and R² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof;

R³ and R⁴ are individually absent or are selected from halogen, alkyl, alkoxy, or hydroxy;

n is 0 or 1;

n² is 0, 1, 2, or 3; and

n³ is 0, 1, 2, or 3.

In some embodiments, R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, wherein R⁷ is a halogen, alkyl, alkoxy, or hydroxy.

In some embodiments, R¹ is an alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, wherein R⁷ is a halogen, alkyl, alkoxy, or hydroxy.

In some embodiments, R² is an alkyl, alkylene-aryl, aryl, alkylene-cycloalkyl, cycloalkyl, alkylene-heterocyclyl, or heterocyclyl, each of which is optionally substituted with one or more R⁸, wherein R⁸ is a halogen, alkyl, alkoxy, or hydroxy.

In other embodiments, the compound can have the formula:

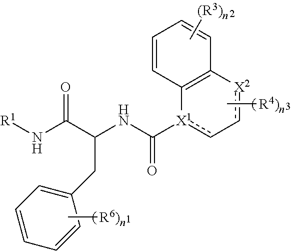

or a pharmaceutically acceptable salt, tautomer, or solvate thereof;

wherein X¹ and X² are each independently N(H) or C(H);

the dashed line is an optional bond;

R¹ is selected from the group hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof;

R³, R⁴, and R⁶ are individually absent or are selected from a halogen, alkyl, alkoxy, or hydroxy;

n¹ is 0, 1, or 3;

n² is 0, 1, 2, or 3; and

n³ is 0, 1, 2, or 3.

In some embodiments, R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, wherein R⁷ is halogen, alkyl, alkoxy, or hydroxy.

In some embodiments, R¹ is an alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, wherein R⁷ is halogen, alkyl, alkoxy, or hydroxy.

In still other embodiments, the compound can have the formula:

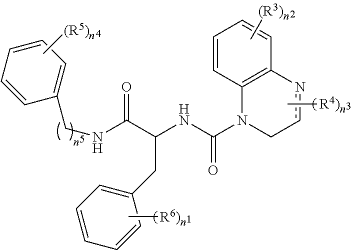

or a pharmaceutically acceptable salt, tautomer, or solvate thereof;

wherein the dashed line is an optional bond;

R³, R⁴, R⁵, and R⁶ are individually absent or are selected from a halogen, alkyl, alkoxy, or hydroxy;

n¹ is 0, 1, or 3;

n² is 0, 1, 2, or 3;

n³ is 0, 1, 2, or 3;

n⁴ is 0, 1, 2, or 3 and

n⁵ is 0, 1, 2, 3, 4, or 5.

In other embodiments, the compound can have the formula:

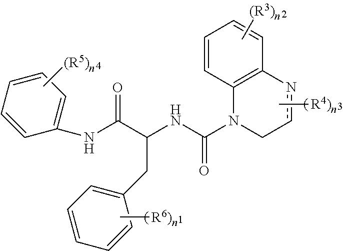

or a pharmaceutically acceptable salt, tautomer, or solvate thereof;

wherein the dashed line is an optional bond;

R³, R⁴, R⁵, and R⁶ are individually absent or are selected from a halogen, alkyl, alkoxy, or hydroxy;

n¹ is 0, 1, or 3;

n² is 0, 1, 2, or 3;

n³ is 0, 1, 2, or 3; and

n⁴ is 0, 1, 2, or 3.

In other embodiment, the compound can have the following formula:

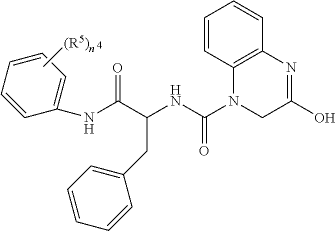

or a pharmaceutically acceptable salt, tautomer, or solvate thereof; wherein

R⁵ is absent or selected from halogen, alkyl, alkoxy, or hydroxy; and

n⁴ is 0, 1, 2, or 3.

In other embodiments, the compound can have the following formula:

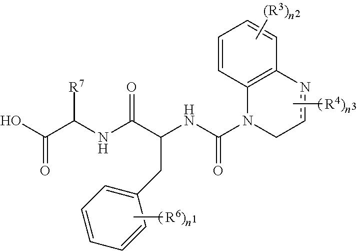

or a pharmaceutically acceptable salt, tautomer, or solvate thereof;

wherein the dashed line is an optional bond;

R³, R⁴, R⁶, and R⁷ are individually absent or are selected from halogen, alkyl, alkoxy, or hydroxy;

n¹ is 0, 1, or 3;

n² is 0, 1, 2, or 3; and

n³ is 0, 1, 2, or 3.

In still other embodiments, can have the following formula:

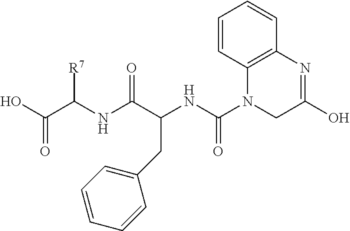

or a pharmaceutically acceptable salt, tautomer, or solvate thereof;

wherein R⁷ is absent or selected from halogen, alkyl, alkoxy, or hydroxy.

In still other embodiments, the compound can have the formula:

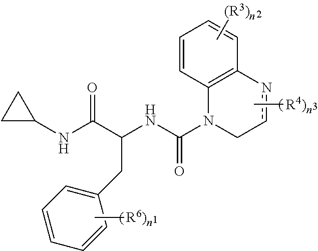

or a pharmaceutically acceptable salt, tautomer, or solvate thereof;

wherein the dashed line is an optional bond;

R³, R⁴, and R⁶ are individually absent or are selected from halogen, alkyl, alkoxy, or hydroxy;

n¹ is 0, 1, or 3;

n² is 0, 1, 2, or 3; and

n³ is 0, 1, 2, or 3.

In other embodiments, the compound can have the following formula:

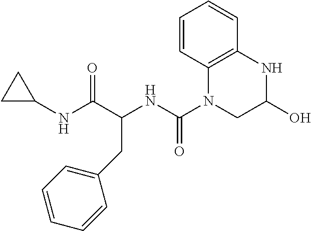 [SMILES: O=C(NC1CC1)C(Cc1ccccc1)NC(=O)N1CC(O)Nc2ccccc21]

or a pharmaceutically acceptable salt, tautomer, or solvate thereof.

In still other embodiments, the compound can have a formula comprising at least one of:

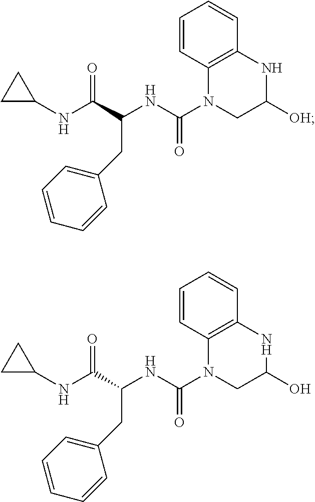

or a pharmaceutically acceptable salt, tautomer, or solvate thereof.

In some embodiments, the compounds can be selected from:

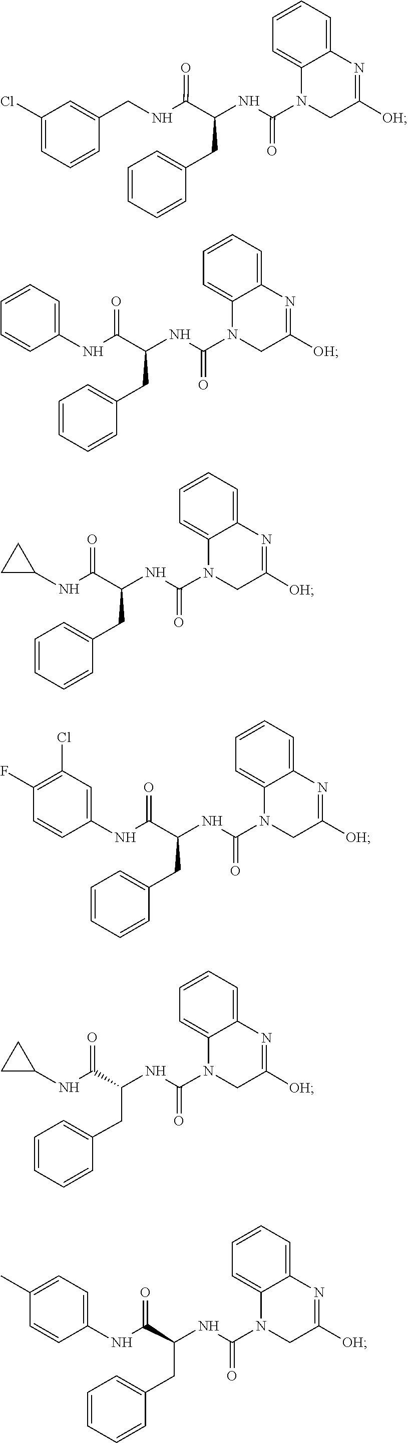 [SMILES: O=C(NCc1cccc(Cl)c1)[C@H](Cc1ccccc1)NC(=O)N1CC(O)=Nc2ccccc21 ; O=C(Nc1ccccc1)[C@H](Cc1ccccc1)NC(=O)N1CC(O)=Nc2ccccc21 ; O=C(NC1CC1)[C@H](Cc1ccccc1)NC(=O)N1CC(O)=Nc2ccccc21 ; O=C(Nc1ccc(F)c(Cl)c1)[C@H](Cc1ccccc1)NC(=O)N1CC(O)=Nc2ccccc21 ; O=C(NC1CC1)[C@@H](Cc1ccccc1)NC(=O)N1CC(O)=Nc2ccccc21 ; Cc1ccc(NC(=O)[C@H](Cc2ccccc2)NC(=O)N2CC(O)=Nc3ccccc32)cc1]

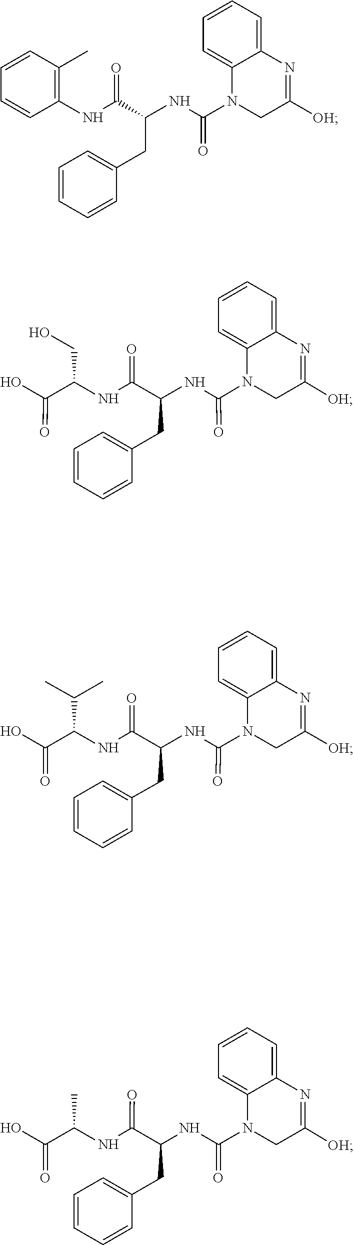 [SMILES: Cc1ccccc1NC(=O)[C@@H](Cc1ccccc1)NC(=O)N1CC(O)=Nc2ccccc21 ; O=C(O)[C@H](CO)NC(=O)[C@H](Cc1ccccc1)NC(=O)N1CC(O)=Nc2ccccc21 ; CC(C)[C@H](NC(=O)[C@H](Cc1ccccc1)NC(=O)N1CC(O)=Nc2ccccc21)C(=O)O ; C[C@H](NC(=O)[C@H](Cc1ccccc1)NC(=O)N1CC(O)=Nc2ccccc21)C(=O)O]

or a pharmaceutically acceptable salt, tautomer, or solvate thereof.

The compounds described herein can be used in methods of treating cancer in a subject. The methods can include administering to the subject therapeutically effective amounts of at least one compounds described above, or a pharmaceutically acceptable salt, tautomer, or solvate thereof.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

More particularly, cancers that may be treated by the compounds, compositions and methods described herein include, but are not limited to, the following: breast cancer, including triple negative breast cancer, cardiac cancers, including, for example sarcoma, e.g., angiosarcoma, fibrosarcoma, rhabdomyosarcoma, and liposarcoma; myxoma; rhabdomyoma; fibroma; lipoma and teratoma; lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma; gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma; genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;liver cancers, including, for example, hepatoma, e.g., hepatocellular carcinoma; cholangiocarcinoma; hepatoblastoma; angiosarcoma; hepatocellular adenoma; and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma; gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma; hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and adrenal gland cancers, including, for example, neuroblastoma.

Cancers may be solid tumors that may or may not be metastatic. Cancers may also occur, as in leukemia, as a diffuse tissue. In certain embodiments, the cancer can be selected from leukemia, breast cancer, prostate cancer, endometrial and non-small cell lung cancer.

Subjects potentially benefiting from the methods described herein include male and female mammalian subjects, including humans, non-human primates, and non-primate mammals. Other mammalian subjects include domesticated farm animals (e.g., cow, horse, pig) or pets (e.g., dog, cat). In some embodiments, the subject can include any human or animal subject who has a disorder characterized by unwanted, rapid cell proliferation of brain cells. Such disorders include, but are not limited to cancers and precancers, such as those described above. For methods of prevention, the subject can include any human or animal subject, and preferably is a human subject who is at risk of obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on.

In certain embodiments, prior to treatment, the patients are selected for having a particular cancer, or for being at risk of a particular cancer. The presence of cancer can be determined by means well known to clinicians. Initial assessment of cancer is based on symptoms presented by the patient. In addition, there are follow-up diagnostic procedures, including, but not limited to PET scans, CAT scans, biopsies, and bio-marker assessments.

Also provided herein are pharmaceutical compositions for the treatment of cancer, comprising a compound or a pharmaceutically acceptable salt form thereof, and a pharmaceutically acceptable carrier or diluent.

While it may be possible for therapeutic compounds described herein to be administered as the raw chemical, it is preferable to present them as a pharmaceutical composition. Pharmaceutical compositions described herein can include a compound described herein, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association one or more therapeutic compounds described above or a pharmaceutically acceptable salt thereof (“active ingredient”) with the carrier which constitutes one or more accessory ingredients. In general, the formulations are prepared by uniformly and intimately bringing into association one or more active ingredients with liquid carriers or finely divided solid carriers or both and then, if necessary, shaping the product into the desired formulation.

Formulations that can be used for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

In some embodiments, a compound described herein can be administered to a subject systemically, (i.e., enteral or parenteral administration). Preparations suitable for oral administration are a solution prepared by dissolving an effective amount of an agent or a pharmaceutically acceptable salt thereof in a diluent such as water, physiological saline, or orange juice; capsules, sachets or tablets comprising an effective amount of one or more therapeutic agents in solid or granular form; a suspension prepared by suspending an effective amount of active ingredient in an appropriate dispersant; an emulsion prepared by dispersing and emulsifying a solution of an effective amount of active ingredient in an appropriate dispersant, and the like.

As preparations suitable for parenteral administration (e.g., intravenous administration, subcutaneous administration, intramuscular administration, topical administration, intraperitoneal administration, intranasal administration, pulmonary administration and the like), aqueous and non-aqueous isotonic sterile injectable liquids are available, which may comprise an antioxidant, a buffer solution, a bacteriostatic agent, an isotonizing agent and the like. Aqueous and non-aqueous sterile suspensions can also be mentioned, which may comprise a suspending agent, a solubilizer, a thickener, a stabilizer, an antiseptic and the like. The preparation can be included in a container such as an ampoule or a vial in a unit dosage volume or in several divided doses. An active ingredient and a pharmaceutically acceptable carrier can also be freeze-dried and stored in a state that may be dissolved or suspended in an appropriate sterile vehicle just before use. In addition to liquid injections, inhalants and ointments are also acceptable. In the case of an inhalant, an active ingredient in a freeze-dried state is micronized and administered by inhalation using an appropriate inhalation device. An inhalant can be formulated as appropriate with a conventionally used surfactant, oil, seasoning, cyclodextrin or derivative thereof and the like as required. In some embodiments, a compound described herein may be incorporated into sustained-release preparations and devices.

In some embodiments, the compounds described herein can be administered to a subject with cancer at an amount effective to inhibit KDM5B, induce HEXIM1 expression, and/or induce p21 expresssion.

The dosage a compound described herein administered to the subject can vary depending on the kind and activity of active ingredient(s), seriousness of disease, animal species being the subject of administration, drug tolerability of the subject of administration, body weight, age and the like, and the usual dosage, based on the amount of active ingredient per day for an adult, can be about 0.0001 to about 100 mg/kg, for example, about 0.0001 to about 10 mg/kg, preferably about 0.005 to about 1 mg/kg. In certain embodiments, dosage can be about 10 mg/kg. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

In another embodiment, the practice of the method in conjunction with additional therapies is contemplated. Additional therapies can include conventional chemotherapy, radiation therapy or surgery directed against solid tumors and for control of establishment of metastases. For example, the administration of therapeutically effective amounts of a compound described herein may be conducted before, during or after chemotherapy, radiation therapy or surgery.

The phrase “combination therapy” embraces the administration of a compound described herein and/or additional further therapies as part of a specific treatment regimen intended to provide a beneficial effect from the co-action of these therapeutic agents.

Administration of these therapeutic agents in combination typically is carried out over a defined time period (usually minutes, hours, days or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents (i.e., a compound described herein), in a substantially simultaneous manner. Substantially simultaneous administration can be accomplished, for example, by administering to the subject a single capsule including a compound described herein having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a third and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

Thus, there is further provided a method of treating cancer comprising administering an effective amount of a compound described herein, or pharmaceutically acceptable salt forms thereof, to a subject, wherein a therapeutically effective amount of one or more additional cancer chemotherapeutic agents are administered to the patient. In some embodiments, a compound described herein or pharmaceutically acceptable salt forms thereof, can restore sensitivity to one or more chemotherapeutic agents in a patient wherein the patient has developed a resistance to the one or more chemotherapeutic agents.

For the purposes of additional cancer chemotherapeutic agent therapy, there are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents. Suitable agents which may be used in combination therapy will be recognized by those of skill in the art.

Exemplary chemotherapeutic agents can include alkylating-type anti-proliferative agents. The alkylating agents are believed to act by alkylating and cross-linking guanine and possibly other bases in DNA, arresting cell division. Typical alkylating agents include nitrogen mustards, ethyleneimine compounds, alkyl sulfates, cisplatin, and various nitrosoureas. A disadvantage with these compounds is that they not only attack malignant cells, but also other cells which are naturally dividing, such as those of bone marrow, skin, gastro-intestinal mucosa, and fetal tissue. Examples of alkylating-type anti-proliferative agents that may be used in the present invention include, but are not limited to, Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromus-tine, Tanabe Seiyaku TA-077, tauromustine, temozolomide, teroxirone, tetraplatin and trimelamol.

In some embodiments, radioprotective agents known to those of skill in the art for radiotherapy may be administered in combination with a compound described herein for the treatment of cancer in a subject. Radiotherapy may include ionizing radiation, particularly gamma radiation irradiated by commonly used linear accelerators or radionuclides. The radiotherapy by radionuclides may be achieved externally or internally. Radiotherapy may include brachytherapy, radionuclide therapy, external beam radiation therapy, thermal therapy (cryoablation hyperthermia), radiosurgery, charged-particle radiotherapy, neutron radiotherapy and photodynamic therapy, and the like.

Radiotherapy can be implemented by using a linear accelerator to irradiate the affected part with X-rays or an electron beam. While the X-ray conditions will differ depending on how far the tumor has advanced and its size and the like, a normal dose will be 1.5 to 3 Gy, preferably around 2 Gy, 2 to 5 times a week, and preferably 4 or 5 times a week, over a period of 1 to 5 weeks, for a total dose of 20 to 70 Gy, preferably 40 to 70 Gy, and more preferably 50 to 60 Gy. While the electron beam conditions will also differ depending on how far the tumor has advanced and its size and the like, a normal dose will be 2 to 5 Gy, preferably around 4 Gy, 1 to 5 times a week, and preferably 2 or 3 times a week, over a period of 1 to 5 weeks, for a total dose of 30 to 70 Gy, and preferably 40 to 60 Gy.

Treatment described herein can also be combined with treatments such as hormonal therapy, proton therapy, cryosurgery, and high intensity focused ultrasound (HIFU), depending on the clinical scenario and desired outcome.

The present invention is illustrated by the following examples. It is to be understood that the particular examples, materials, amounts, and procedures are to be interpreted broadly in accordance with the scope and spirit of the invention as set forth herein.

### EXAMPLE 1

We elucidated the regulatory networks by which HEXIM1 protein (hexamethylene bisacetamide induced protein 1) functions as a tumor suppressor of breast cancer and other solid tumor cancers. This has led to the development of a promising, potent and selective compound series which function as inhibitors of the epigenetic modulator enzyme Lysine Demethylase KDM5B. Our work has primarily focused on Triple Negative Breast Cancer due to the scarcity of effective medicines for this subset of patients and unlike the results shown with other KDM5B inhibitors we are seeing an effect upon proliferation of TNBC cell lines in the very low nanomolar range.

KDM5B has been the target of many attempts at drug development and the approach of developing competitive inhibitors of 2-oxoglutarate (2-OG) has proven surprisingly difficult. This is perhaps best explained as challenging battle to defeat very high levels of 2-OG within cells and the evidence that in cancerous cells not only do 2-OG levels rise further, but even higher levels of the oncometabolite and 2-OG analog 2-hydroxyglutarate also appear. Consequently, we have pursued alternate binding modes for prototype small molecule pharmaceuticals within the KDM5B JmJC domain. We have additionally shown that the anticancer effects of KDM5B inhibition, rather than being a global indirect effect of epigenetic modulation, is perhaps mediated entirely through the re-expression of HEXIM1 in cancer cells.

### Binding Affinity Determination and Generation of Crystal Structures of KDM5B JmjC Catalytic Domain in Complex With Our Best Lead Compound(s)

While we have been able to model (molecular docking) using in silico data of existing inhibitor/KDM5B JmJC crystal complexes, the fine tuning of our leads will greatly benefit from the SPR analyses of binding affinities and generation of a crystal structure complex between the binding target KDM5B JmjC and lead compound(s). We have successfully expressed and purified the target protein and it is also available commercially.

### Crystallization and Molecular Biophysics Core, (PEPCMBC)

Crystallization trials using the protein enzyme sample will be performed in hanging-drop and sitting-drop formats using a number of diverse sparse matrix and customized crystallization screens. We will use a Phoenix crystallization robot to enable fast and automated screening of about 1,000 initial crystallization conditions, with 200 nL of protein solution per condition. There are many structures of KDM5B JmJC complexed with inhibitors available in PDB. Unlike other groups however, we will include 2-oxoglutarate (2-OG) in crystallization media so that our compounds find their biologically relevant binding pose.

Compositions for the Inhibition of Histone Demethylases and/or the Induction of Tumor Suppressor HEXIM1

The following structures as either enantiomers or as racemic mixtures have been shown as novel inhibitors of KDM5B and inducers of HEXIM1 using cultured human cancer cells such a triple negative breast cancer cell line MDA-MB-231 or prostate cancer cell line PC3. Doses as low as 0.5 to 5 nanomolar achieved KDM5B inhibition/HEXIM induction, which is an unprecedented level of potency when dosing cells (in contrast to inhibition values reported in biochemical assays).

TABLE 1

| | | |
|---|---|---|
| 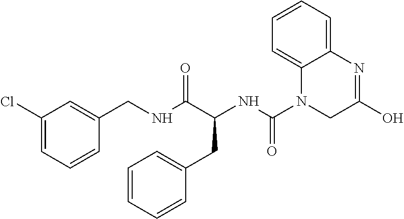 [SMILES: O=C(NCc1cccc(Cl)c1)[C@H](Cc1ccccc1)NC(=O)N1CC(O)=Nc2ccccc21] | 1 | (2S)-N-[(3-chlorophenyl)methyl]-2- (13-hydroxy-1,2- dihydroquinoxaline-1- carbonyl)amino]-3- phenylpropanamide |
| 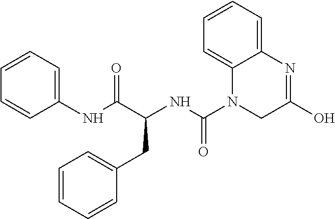 [SMILES: O=C(Nc1ccccc1)[C@H](Cc1ccccc1)NC(=O)N1CC(O)=Nc2ccccc21] | 2 | (2S)-2-[(3-hydroxy-1,2- dihydroquinoxaline-1- carbonyl)omino[-N,3- diphenylproponomide |
| 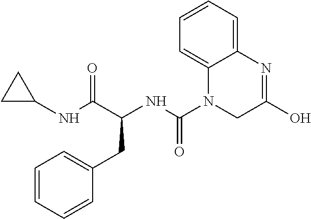 [SMILES: O=C(NC1CC1)[C@H](Cc1ccccc1)NC(=O)N1CC(O)=Nc2ccccc21] | 3 | (12S)-N-cyclopropyl-2-[(3-hydroxy- 1,2-dihydroquino xaline-1- carbonyl)amino]-3- phenylpropanomide |
| 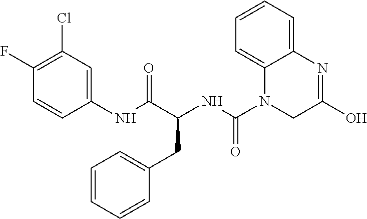 [SMILES: O=C(Nc1ccc(F)c(Cl)c1)[C@H](Cc1ccccc1)NC(=O)N1CC(O)=Nc2ccccc21] | 4 | (2S)-N-(3-chloro-4-fluorophenyl)-2- [(3-hydroxy-1,2-dihydroquinoxaline- 1-carbanyl)amino]-3- phenylpropanamide |
| 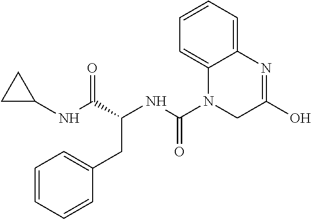 [SMILES: O=C(NC1CC1)[C@@H](Cc1ccccc1)NC(=O)N1CC(O)=Nc2ccccc21] | 5 | N-cyclapropyl-2-[(3-hydroxy-1,2- dihydroquinoxoline-1- carbanyl)amino]-3- phenylpropanamide (racemic) |
| 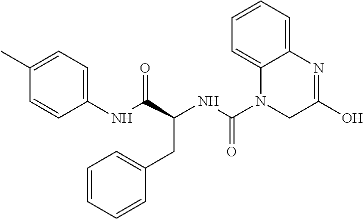 [SMILES: Cc1ccc(NC(=O)[C@H](Cc2ccccc2)NC(=O)N2CC(O)=Nc3ccccc32)cc1] | 6 | 2-[(3-hydroxy,-1,2- dihydroquinoxaline-1- carbonyl)amino]-N-(4- methylphenyl)-3-phenylpropanamide (racemic?) |
| 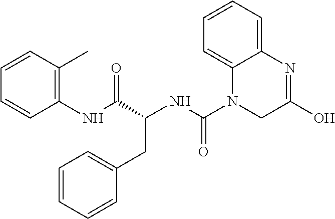 [SMILES: Cc1ccccc1NC(=O)[C@@H](Cc1ccccc1)NC(=O)N1CC(O)=Nc2ccccc21] | 7 | 2-[(3-hydroxy-1,2- dihydroquinoxaline-1- carbonyl)amino]-N-(2- methylphenyl)-3-phenylpropanamide (racemic) |
| 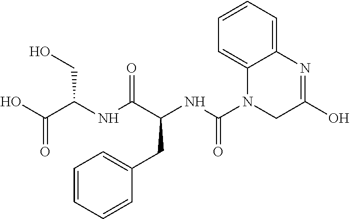 [SMILES: O=C(O)[C@H](CO)NC(=O)[C@H](Cc1ccccc1)NC(=O)N1CC(O)=Nc2ccccc21] | 8 | (2S)-3-hydroxy-2-[(2S)-2-[(3- hydroxy-1,2-dihydroquinoxaline-1- carbonyl)amino]-3- phenylpropanamido]propanoic acid |
| 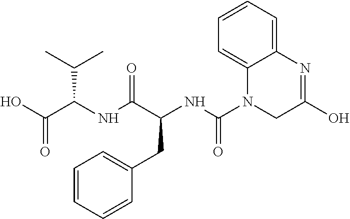 [SMILES: CC(C)[C@H](NC(=O)[C@H](Cc1ccccc1)NC(=O)N1CC(O)=Nc2ccccc21)C(=O)O] | 9 | (2S)-2-[(2S)-2-[[3-hydroxy-1,2- dihydroquinoxaline-1- carbonyl)amino]-3- phenylpropanomido]-3- methylbutanoic acid |
| 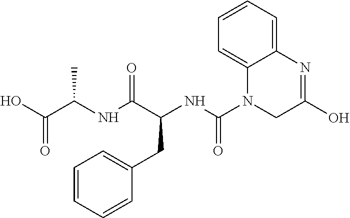 [SMILES: C[C@H](NC(=O)[C@H](Cc1ccccc1)NC(=O)N1CC(O)=Nc2ccccc21)C(=O)O] | 10 | (2S)-2-[(2S)-2-[[3-hydroxy-1,2- dihydroquinoxaline-1- carbonyl)amino]-3- phenylpropanamido]propanoic acid |

### In Vitro ADME/Tox Screening and Closed Loop of Compound Improvement.

Our lead series compounds have generally favorable predicted pharmacological properties, in particular compliance with Lipinski Rule of Five (R05), and logP, logD and logS values that are compatible with an orally bioavailable drug. However full PK/PD and in vitro ADME/Tox profiles of the leads need to be established and then improved through substitution chemistry. PK/PD measurements are available on a fee for service basis from CWRU Pharmacology core. For other services we will employ third party CRO organizations (such as AMRI) to provide non-GLP ADME/Tox testing. A standard battery of in vitro assays will be employed in each pass of the iterative loop process:

TABLE 2

| in vitro Assays | |
|---|---|
| ADME | Kinetic |
| | Thermostability |
| | Metabolic stability |
| | Caco-2 permeability |
| | P450 inhibition (5 in 1) |
| Cardiotox | hERG |
| | NaV1.5 |
| | Cardiac test panel |
| Hepatotoxicity | Hepatotoxicity |
| | Hepatolipidosis |
| Genotoxicity | Ames assay |

Improved compounds will also be tested in functional assays on TNBC cell lines, measuring efficacy of KDM5B inhibition (appearance of H3K4me3/2 marks on the HEXIM1 promoter), induction of HEXIM1 protein (Western blot), induction of quiescence (Western blot analyses of p21 and cell cycle analyses), and stem cell fraction (flow cytometry and mammospheres). IC50 will be measured using an AlphaScreen type biochemical assay with H3K4me3/2 substrate via a third party service provider.

### EXAMPLE 2

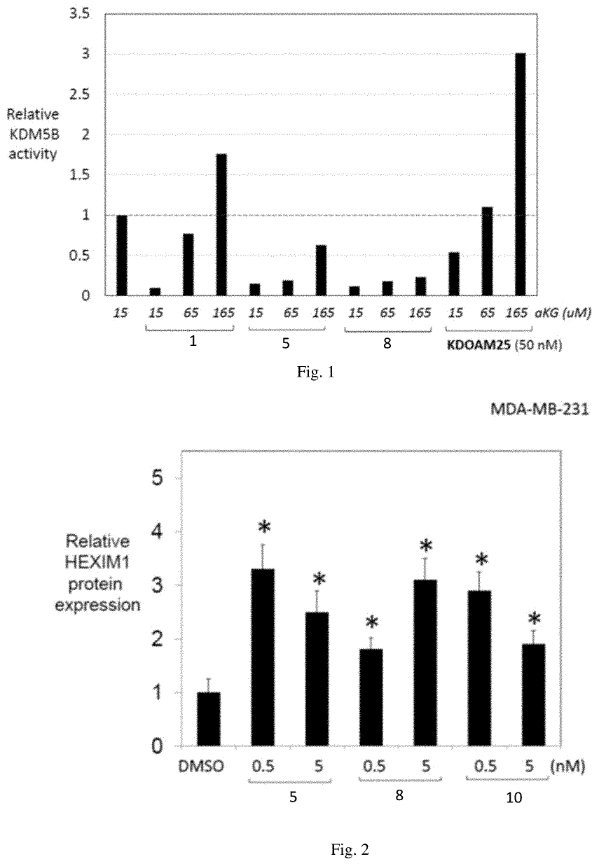

We have recently optimized a commercial JARID1B (KDM5B) Chemiluminescent Assay Kit from BPS Bioscience (Cat #50517). We increased αKG/2-OG from 5 μM to 15 μM in the assay buffer to increase signal to noise ratio. KDM5B Km for αKG is reported in the literature as 9 μM. We then tested known competitive inhibitor KDOAM25 at its approximate IC₅₀ of 50 nM against 15, 65 or 165 μM αKG. (FIG. 1). We found that as expected, αKG is rate limiting in the assay, thus additional αKG dramatically boosts signal (demethylated substrate). However above 165 μM αKG, the assay broke down—perhaps the increasing acidity broke down the buffering. We also reproduced the results of others where higher levels of αKG relieve the inhibition of KDM5B by KDOAM25.

Using this same modified assay format, we tested selected members of our lead chemical series, compound 1, compound 5, and compound 8 at 50 nM (for direct comparison to KDOAM25) in the presence of 15, 65 or 165 μM αKG. Compound 5 and compound 8 in particular appear to resist the relief of inhibition by excess αKG at 165 μM αKG, strongly suggesting that these inhibitors are both more potent than KDOAM25 and also much less sensitive to competition from KG. One caveat is that compound 5 is a racemic mixture in unknown ratio of S/R enantiomers, so the R enantiomer may perform better in this assay than shown without competition from the S enantiomer. Equally compound 1 is an S enantiomer, so its R enantiomer might bind with less competition with αKG. Interestingly compound 8 is an S enantiomer and shows little if any competition from αKG.

Most of our work characterizing compounds has used cellular assays since we have been working with HEXIMlinducing compounds long before we identified KDM5B as the pharmacological target. We use Western Blot analysis of cultured cancer cell lines exposed to various concentrations of compound and look for increased expression of HEXIM1 and p21 (a marker of quiescence/G₀). FIG. 2 shows the induction of HEXIM1 protein in MDA-MB-231 Triple Negative Breast Cancer (TNBC) cells using compounds compound 5, compound 8 or compound 10 at 0.5 or 5 nM. All members of the lead chemical series, compound 1, and compound 2 through compound 10 are able to induce HEXIM1 expression in this cell line at these concentrations.

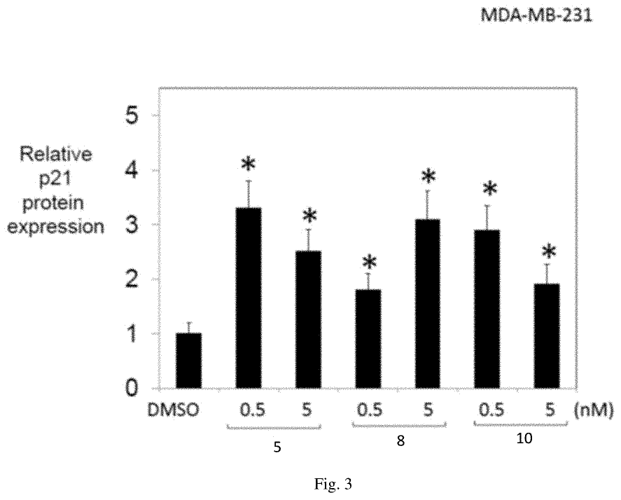

In the context of treating cancer, we discovered that while all members of the lead chemical series are able to induce HEXIM1 expression, only a subset are effective at also inducing p21 (the marker of quiescence/Go), in MDA-MB-231 and other TNBC cell lines. (FIG. 3).

Since ideal oncology medicines would limit or inhibit proliferation, we consider p21 induction an important characteristic among the lead chemical series. Cancer cells are typically mutated for p53, which is normally the positive regulator of p21. Consequently, other pathways, such as PI3K/AKT, may be transiently necessary for p21 induction when HEXIM1 is induced through KDM5B inhibition. We speculate that some of our lead compound series may have some off target activity within the PI3K/AKT pathway and thus blunt the induction of p21. We recognize that some of these effects may vary according to the S/R enantiomer used and correlating p21 induction to specific structural features such as chirality needs to be investigated more thoroughly.

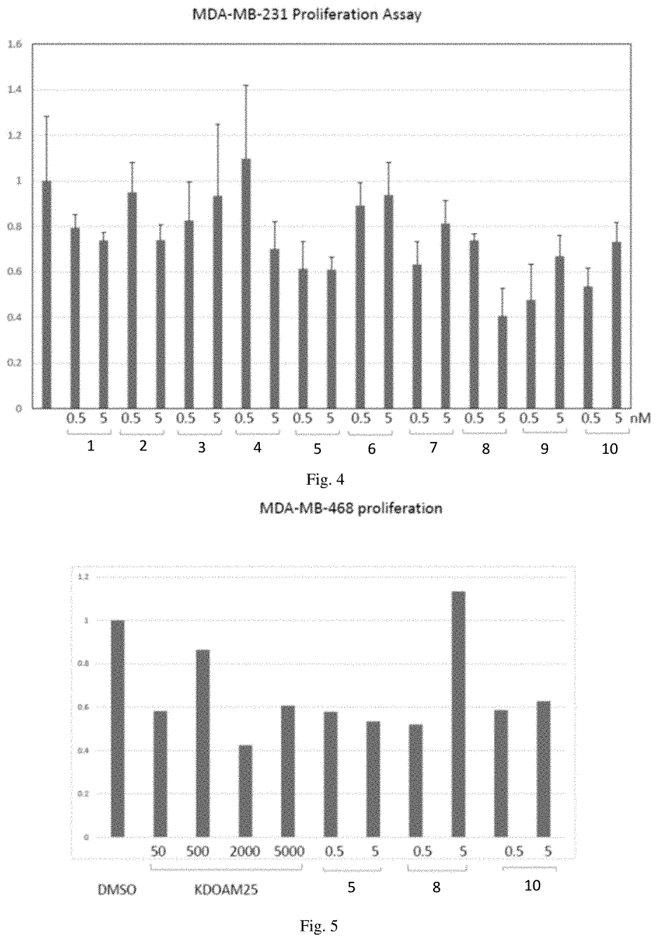

The overall impact of HEXIM1 induction combined with an effective induction of p21 can be seen in the results of a proliferation assay using TNBC MDA-MB-231 and all the members of the lead chemical series (FIGS. 4) at 0.5 and 5 nM doses.

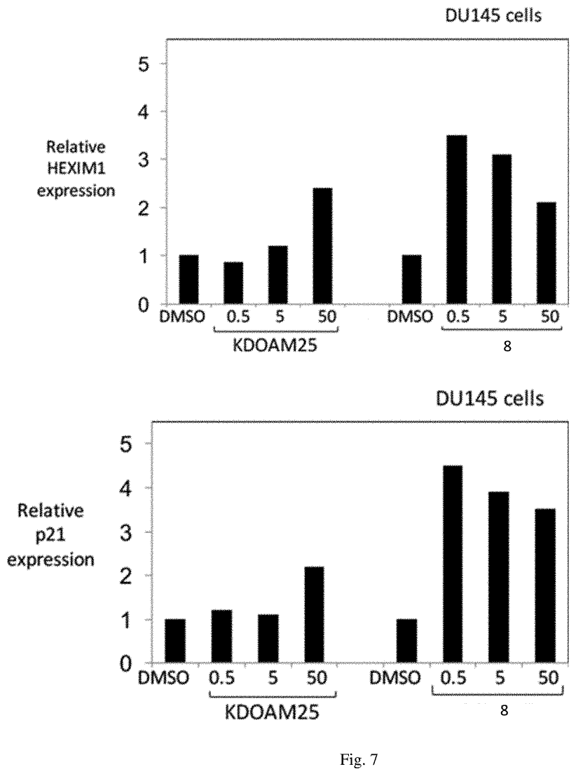

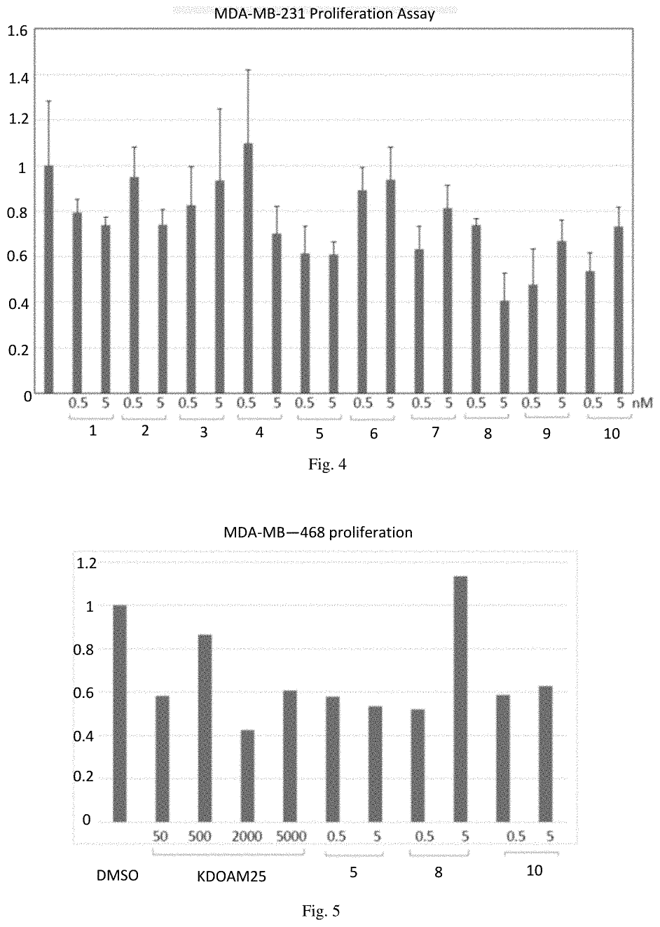

We have also shown similar results using the TNBC MDA-MB-468 cell line and comparing some members of the lead compound series against much higher doses of KDOAM25. (FIG. 5, doses are given in nM). The antiproliferative effects of the lead chemical series have also been observed against prostate cancer cell lines such as PC3 and C4-2 and are generally more pronounced (FIG. 7), suggesting that HEXIM1 induction via inhibition of KDM5B (with accompanying p21 induction) may be a useful approach to other solid tumor cancers besides breast.

With regard to the potency and selectivity of the lead compound series, using the same modified JARID1B/KDM5B Chemiluminescent assay (15 μM αKG) mentioned earlier, we observed 75% inhibition of KDM5B using 50 pM concentrations of compound 8, which implies a potential pIC50 of 10.3 or better. Similar results were obtained with certain of the other lead series compounds and we are continuing to refine these results. Compound 8 is a little more soluble than other members of the series. However, the initial results look promising, and perhaps not totally surprising for compounds with efficacy on cultured cells at 0.5 to 5 nM doses. Once again, the influence of enantiomers on potency still needs to be evaluated.

With regard to selectivity for KDM5B, the initial in silico compound screening focused on targeting a theoretical putative binding site within the JmjC catalytic core of KDM5B but away from the αKG binding pocket. Compounds which scored very highly (G>10.0) were then screened in silico against the cognate region of KDM4A and compounds with predicted preferred affinity for KDM5B were selected. Confirming selectivity for KDMS over other KDMs can be demonstrated using other kits similar to the JARID1B/KDM5B Chemiluminescent Assay Kit, though we will adopt an AlphaScreen type format since we will have more options and assay flexibility. KDM4A/B and KDM6B in particular will be good enzymes to test for compound selectivity. In terms of KDM5B selectivity over KDMSA/C/D—again an AlphaScreen type format can also be employed to show the degree of preference for KDM5B. In addition, off target chemical profiling and fine SAR work from a crystal structure developed in Year 1 of the project will help to tune or enhance the KDM5B selectivity once we have confirmed the binding modality. While the JmjC domain is conserved among KDMSA/B/C/D there is sufficient variation to create or enhance preferential selectivity for kDM5B over other KDMS family members.

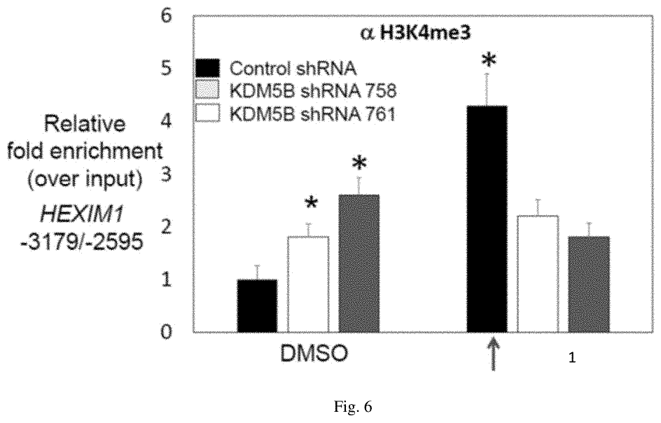

With respect to functional data, we have shown that only KDM5B is necessary to induce re-expression of HEXIM1 via selective KDM5B shRNA knockdown. KDMSA/C/D appear to compensate poorly, if at all for the loss of KDM5B in terms of the accumulation of H3K4me3 in the HEXIM1 promoter. FIG. 6 shows this result using a ChIP assay on the —3179/—2595 region of the HEXIM1 promoter and shows that inhibitor compound 1 likewise causes a particularly strong accumulation of H3K4me3 on the promoter (FIG. 6 Control shRNA/44 dosing combination, indicated with red arrow).

The following table illustrates a table showing predicted chemical properties that illustrate the relative quality of the lead chemical series. All members are RO5 compliant.

TABLE 3

| | | | logP (wildman- | | |
|---|---|---|---|---|---|
| Compound | MW | Enantiomer | crippen) | LogS | logD_{7.4} |
| 1 | 462.926 | S | 4.385 | −5.405 | 1.057 |
| 2 | 414.455 | S | 4.054 | −5.327 | 0.905 |
| 3 | 378.424 | S | 2.694 | −4.092 | 0.304 |
| 4 | 466.889 | S | 4.847 | −5.677 | 1.028 |
| 5 | 378.424 | racemic | 2.694 | −4.092 | 0.304 |
| 6 | 428.482 | racemic | 4.363 | −5.51 | 1.017 |
| 7 | 428.482 | racemic | 4.363 | −5.504 | 1.014 |
| 8 | 426.422 | S | 0.977 | −3.623 | −0.32 |
| 9 | 438.476 | S | 2.641 | −4.091 | 0.319 |
| 10 | 410.422 | S | 2.005 | −4.017 | −0.019 |

FIG. 7 is a graph illustrating prostate cancer cell line DU145 cells treated with 0.5 nM, 5 nM or 50 nM of either compound KDOAM25 (IC50˜42 nM) or compound 8.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention the following is claimed:

1. A compound having the following formula: 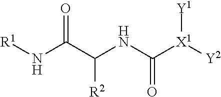
or a pharmaceutically acceptable salt, tautomer, or solvate thereof; wherein
X¹ is N(H) or C(H); and
R¹, R², Y¹, and Y² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, or a substituted or unsubstituted heterocyclyl, and wherein the compound inhibits KDM5B, induces expression of HEXIM1, and/or expression of p21.

2. The compound of claim 1, wherein is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

3. The compound of claim 1, wherein R¹ is a alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

4. The compound of claim 1, wherein R² is an alkyl, alkylene-aryl, aryl, alkylene-cycloalkyl, cycloalkyl, alkylene-heterocyclyl, or heterocyclyl, each of which is optionally substituted with one or more R⁸, and wherein R⁸ is halogen, alkyl, alkoxy, or hydroxy.

5. The compound of claim 1, wherein Y¹ and Y² are linked to form a cyclic or polycyclic ring, wherein the ring is an aryl, a heteroaryl, a cycloalkyl, or a heterocyclyl, each of which is optionally substituted with one or more R⁹, and wherein R⁹ is halogen, alkyl, alkoxy, or hydroxy.

6. The compound fo claim 1, having the following formula: 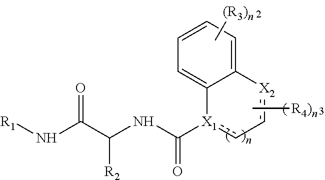
or a pharmaceutically acceptable salt, tautomer, or solvate thereof;
wherein X¹ and X² are each independently N or C(H);
the dashed line is an optional bond;
R¹ and R² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof;
R³ and R⁴ are individually absent or are selected from halogen, alkyl, alkoxy, or hydroxy;
n is 0 or 1;
n² is 0, 1, 2, or 3; and
n³ is 0, 1, 2, or 3.

7. The compound of claim 6, wherein R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

8. The compound of claim 6 wherein R¹ is a alkylene-carboxy, alkylene(hydroxyl)-carboxy, alkylene(alkyl)-carboxy, alkylene-aryl, aryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

9. The compound of claim 6, wherein R² is an alkyl, alkylene-aryl, aryl, alkylene-cycloalkyl, cycloalkyl, alkylene-heterocyclyl, or heterocyclyl, each of which is optionally substituted with one or more R⁸, wherein R⁸ is halogen, alkyl, alkoxy, or hydroxy.

10. The compound of claim 1, selected from: 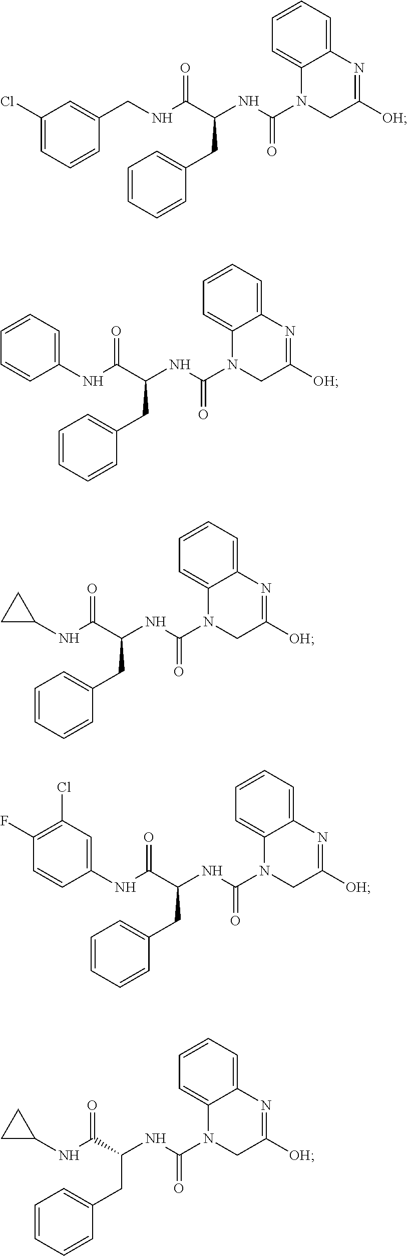 [SMILES: O=C(NCc1cccc(Cl)c1)[C@H](Cc1ccccc1)NC(=O)N1CC(O)=Nc2ccccc21 ; O=C(Nc1ccccc1)[C@H](Cc1ccccc1)NC(=O)N1CC(O)=Nc2ccccc21 ; O=C(NC1CC1)[C@H](Cc1ccccc1)NC(=O)N1CC(O)=Nc2ccccc21 ; O=C(Nc1ccc(F)c(Cl)c1)[C@H](Cc1ccccc1)NC(=O)N1CC(O)=Nc2ccccc21 ; O=C(NC1CC1)[C@@H](Cc1ccccc1)NC(=O)N1CC(O)=Nc2ccccc21] 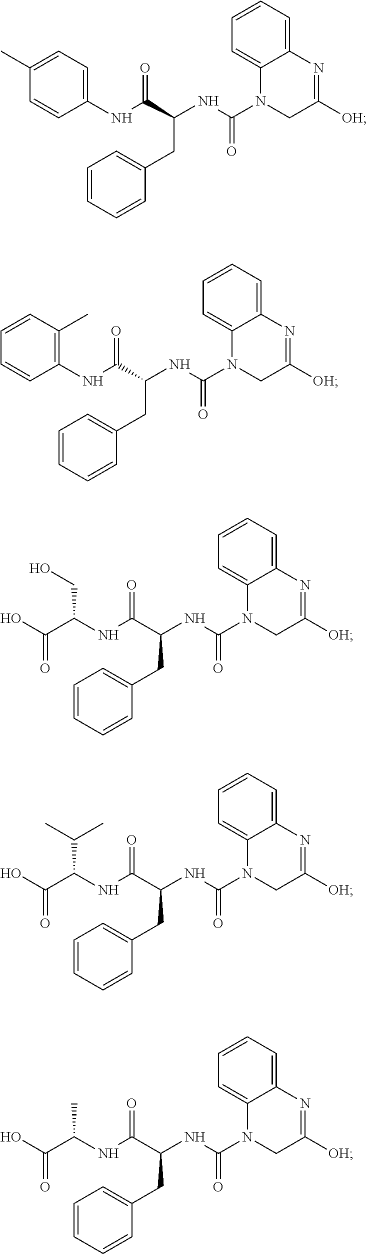 [SMILES: Cc1ccc(NC(=O)[C@H](Cc2ccccc2)NC(=O)N2CC(O)=Nc3ccccc32)cc1 ; Cc1ccccc1NC(=O)[C@@H](Cc1ccccc1)NC(=O)N1CC(O)=Nc2ccccc21 ; O=C(O)[C@H](CO)NC(=O)[C@H](Cc1ccccc1)NC(=O)N1CC(O)=Nc2ccccc21 ; CC(C)[C@H](NC(=O)[C@H](Cc1ccccc1)NC(=O)N1CC(O)=Nc2ccccc21)C(=O)O ; C[C@H](NC(=O)[C@H](Cc1ccccc1)NC(=O)N1CC(O)=Nc2ccccc21)C(=O)O]
or a pharmaceutically acceptable salt, tautomer, or solvate thereof.

11. A method treating cancer in a subject in need thereof, the method comprising:
administering to the subject a therapeutically effective amount of a compound having the following formula: 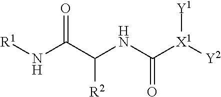
or a pharmaceutically acceptable salt, tautomer, or solvate thereof; wherein
X¹ is N(H) or C(H); and
R¹, R², Y¹, and Y² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, or a substituted or unsubstituted heterocyclyl.

12. The method of claim 11, wherein is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

13. The method of claim 11, wherein R² is an alkyl, alkylene-aryl, aryl, alkylene-cycloalkyl, cycloalkyl, alkylene-heterocyclyl, or heterocyclyl, each of which is optionally substituted with one or more R⁸, and wherein R⁸ is halogen, alkyl, alkoxy, or hydroxy.

14. The method of claim 11, wherein Y¹ and Y² are linked to form a cyclic or polycyclic ring, wherein the ring is an aryl, a heteroaryl, a cycloalkyl, or a heterocyclyl, each of which is optionally substituted with one or more R⁹, and wherein R⁹ is halogen, alkyl, alkoxy, or hydroxy.

15. The method of claim 11, wherein the compound has the following formula: 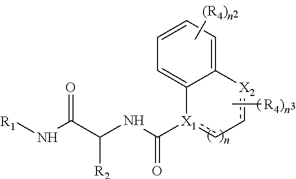
or a pharmaceutically acceptable salt, tautomer, or solvate thereof;
wherein X¹ and X² are each independently N or C(H);
the dashed line is an optional bond;
R¹ and R² are independently selected from the group consisting of hydrogen, substituted or unsubstituted alkyl, alkenyl, alkynyl, aryl, heterocycloalkenyl, heteroaryl or heterocyclyl, alkaryl, aralkyl, halo, silyl, hydroxyl, sulfhydryl, alkoxy, alkenyloxy, alkynyloxy, aryloxy, acyl, acyloxy, alkoxycarbonyl, aryloxycarbonyl, alkylcarbonato, arylcarbonato, carboxy, carboxylato, carbamoyl, alkyl-carbamoyl, arylcarbamoyl, thiocarbamoyl, carbamido, cyano, isocyano, cyanato, isocyanato, isothiocyanato, azido, formyl, thioformyl, amino, alkyl amino, alkyl amino substituted with hydroxyl, aryl amino, alkylamido, arylamido, sulfanamido, imino, alkylimino, arylimino, nitro, nitroso, sulfo, sulfonato, alkylsulfanyl, arylsulfanyl, C₁-C₂₄ alkylsulfinyl, arylsulfinyl, alkylsulfonyl, arylsulfonyl, sulfonamide (—SO₂—NH₂, —SO₂NY₂ (wherein Y is independently H, arlyl or alkyl), phosphono, phosphonato, phosphinato, phospho, phosphino, polyalkyl ethers, phosphates, phosphate esters, groups incorporating amino acids or other moieties expected to bear positive or negative charge at physiological pH, and combinations thereof;
R³ and R⁴ are individually absent or are selected from halogen, alkyl, alkoxy, or hydroxy;
n is 0 or 1;
n² is 0, 1, 2, or 3; and
n³ is 0, 1, 2, or 3.

16. The method of claim 15, wherein R¹ is an alkyl, alkoxy, alkylene-carboxy, alkylene(hydroxyl)-carboxy, alkylene(alkyl)-carboxy, alkylene-aryl, aryl, alkylene-heteroaryl, heteroaryl, alkylene-cycloalkyl, cycloalkyl, alkylene-heterocyclyl, or heterocyclyl, each of which is optionally substituted with one or more R⁷, and wherein R⁷ is halogen, alkyl, alkoxy, or hydroxy.

17. The method of claim 15, wherein R² is an alkyl, alkylene-aryl, aryl, alkylene-cycloalkyl, cycloalkyl, alkylene-heterocyclyl, or heterocyclyl, each of which is optionally substituted with one or more R⁸, wherein R⁸ is halogen, alkyl, alkoxy, or hydroxy.

18. The method of claim 11, wherein the compound is selected from: 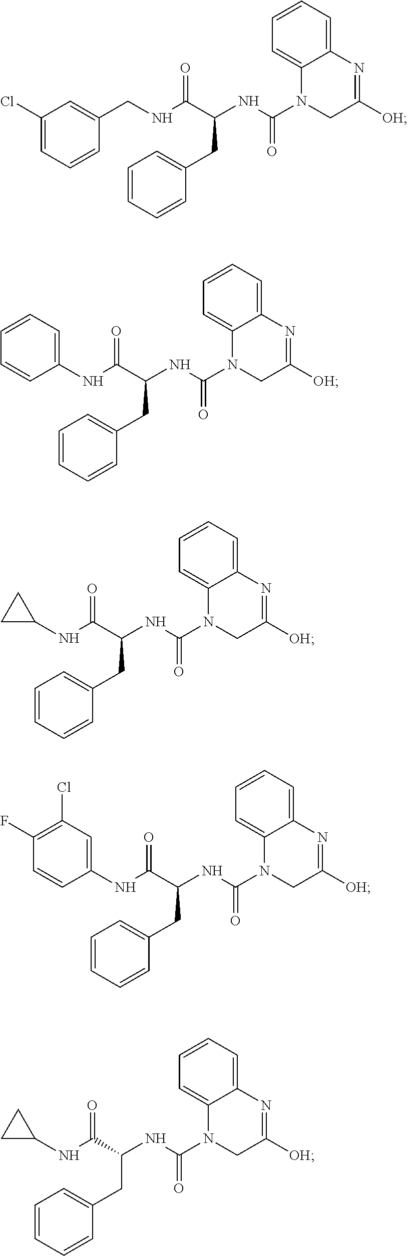 [SMILES: O=C(NCc1cccc(Cl)c1)[C@H](Cc1ccccc1)NC(=O)N1CC(O)=Nc2ccccc21 ; O=C(Nc1ccccc1)[C@H](Cc1ccccc1)NC(=O)N1CC(O)=Nc2ccccc21 ; O=C(NC1CC1)[C@H](Cc1ccccc1)NC(=O)N1CC(O)=Nc2ccccc21 ; O=C(Nc1ccc(F)c(Cl)c1)[C@H](Cc1ccccc1)NC(=O)N1CC(O)=Nc2ccccc21 ; O=C(NC1CC1)[C@@H](Cc1ccccc1)NC(=O)N1CC(O)=Nc2ccccc21] 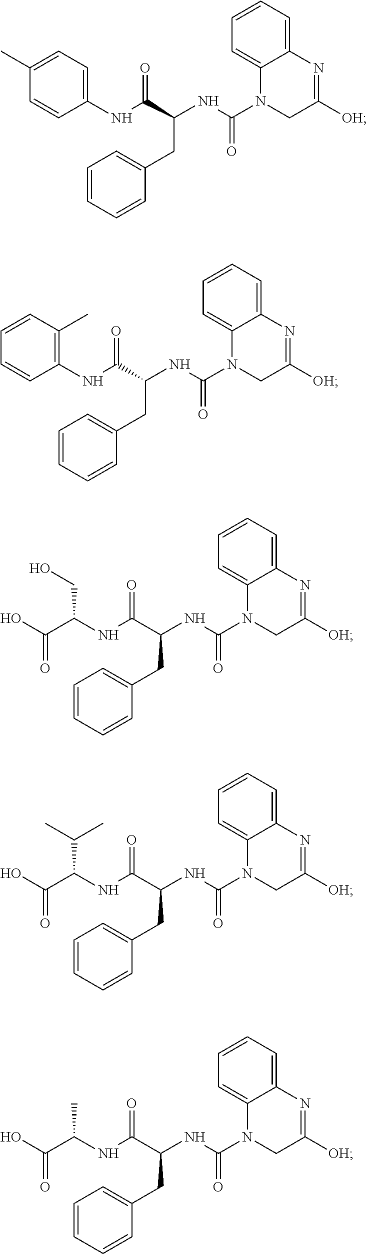 [SMILES: Cc1ccc(NC(=O)[C@H](Cc2ccccc2)NC(=O)N2CC(O)=Nc3ccccc32)cc1 ; Cc1ccccc1NC(=O)[C@@H](Cc1ccccc1)NC(=O)N1CC(O)=Nc2ccccc21 ; O=C(O)[C@H](CO)NC(=O)[C@H](Cc1ccccc1)NC(=O)N1CC(O)=Nc2ccccc21 ; CC(C)[C@H](NC(=O)[C@H](Cc1ccccc1)NC(=O)N1CC(O)=Nc2ccccc21)C(=O)O ; C[C@H](NC(=O)[C@H](Cc1ccccc1)NC(=O)N1CC(O)=Nc2ccccc21)C(=O)O]
or a pharmaceutically acceptable salt, tautomer, or solvate thereof.

19. The method of claim 11, wherein the cancer treated is breast cancer.

20. The method of claim 11, wherein the cancer treated is triple negative breast cancer.
